(19) United States
(12) Patent Application Publication
(10) Pub. No.: US 2024/0216492 A1
(43) Pub. Date: Jul. 4, 2024

(54) USE OF ONCOLYTIC ADENOVIRUS FOR THE TREATMENT OF PEDIATRIC BRAIN CANCER

(71) Applicants: DNATRIX, INC., San Diego, CA (US); UNIVERSIDAD DE NAVARRA, Pamplona (ES)

(72) Inventors: Erin MITCHELL, San Diego, CA (US); Frank TUFARO, San Diego, CA (US); Marta ROLDÁN, Pamplona (ES); Sonia TEJADA-SOLIS, Pamplona (ES); Jaime PÉREZ DE LARRAYA, Pamplona (ES)

(21) Appl. No.: 18/557,339

(22) PCT Filed: Apr. 26, 2022
(86) PCT No.: PCT/US2022/026392

§ 371 Date: Oct. 26, 2023

Related U.S. Application Data
(60) Provisional application No. 63/179,899, filed on Apr. 26, 2021.

(51) Int. Cl. A61K 39/00; A61K 45/06; A61N 5/10; A61P 35/00; C12N 15/86

(52) U.S. Cl. CPC A61K 39/001188; A61K 39/001104; A61K 45/06; A61N 5/10; A61P 35/00; C12N 15/86; A61K 2039/5256; A61K 2039/54; A61K 2039/545; A61N 2005/1098; C12N 2710/10322; C12N 2710/10334; C12N 2710/10343; C12N 2710/10362; C12N 2710/10371

(57) ABSTRACT

The disclosure relates to the use of oncolytic adenoviruses for the treatment of pediatric brain tumors, such as, for example, diffuse intrinsic pontine glioma (DIPG).

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### BACKGROUND

### Field

The present disclosure relates generally to the fields of oncology and cancer therapy. More particularly, it concerns replicative oncolytic viruses and their use to treatment of pediatric brain cancer.

### Description of Related Art

Brain tumors are the leading cause of cancer death in children. The incidence of newly diagnosed brain tumors in the pediatric population is 3.3 cases per 100,000 children every year. Pediatric brain tumors are biologically distinct from brain tumors in adults. There are key genetic and epigenetic differences in pediatric brain tumors that are associated with ages of onset, anatomical distribution, clinical outcome, and histopathological and radiological features. In adults, supratentorial high-grade glioma (WHO-Grade IV), also known as glioblastoma, is the most common primary brain tumor. In contrast, over 60% of pediatric brain tumors are infratentorial, occurring below the tentorium in the cerebellum or brainstem, and pediatric gliomas are more often low grade (WHO-Grade I-II). In addition, brainstem gliomas are exceedingly rare in adults, while 10-15% of pediatric brain tumors are localized to the brainstem. Over 50% of pediatric brainstem tumors are brainstem gliomas.

Brainstem Gliomas are classified into four categories on the basis of anatomic location and radiographic appearance: diffuse, focal intrinsic, focal exophytic, and cervicomedullary. Of these, the most common form is Diffuse Intrinsic Pontine Glioma (DIPG), accounting for 80% of all brainstem gliomas.

Currently, there are no effective treatments for DIPG; these tumors are not amenable to surgical resection and diagnosis is based on characteristic appearance on MR Imaging. Clinical trials of chemotherapeutics and targeted agents extrapolated from adult GBM studies have failed to show a survival benefit (Mackay 2017). As such, improved therapies for DIPG are desperately in need.

### SUMMARY

The present disclosure relates generally to compositions and methods for treating subjects with pediatric brain tumors comprising administering to said subject a replication competent oncolytic adenovirus. Within certain embodiments, the brain tumor is a glioma, such as a Diffuse Intrinsic Pontine Glioma (or “DIPG”) or a pediatric high-grade glioma. Other representative pediatric brain tumors include Atypical Teratoid/Rhabdoid Tumors (“AT/RT”) and Primitive Neuroectodermal Tumors (“CNS-PNET”).

Within certain embodiments of the invention, the pediatric patient has one or more of a H3.3K27M, H3.1K27M, H3.2K27M, wild type H3, H3.3G34RV, and/or p53 mutation (see generally, Mackay et al, 2017 Cancer Cell 32:520-537, Oct. 9, 2017, which is incorporated by reference in its entirety).

Within preferred embodiments of the invention, the subject is treated with an oncolytic adenoviral vector (e.g., an adenoviral vector comprising Delta-24 or Delta-24-RGD). Within yet further embodiments of the invention, the subject is treated with an agent that produces a Th1 phenotype (e.g., a PD-1/PD/L1 receptor antagonist such as for example, MDC-1106, MK-3475 (pembrolizumab), AMP-224, Pidilizumab and MDX-1105.

Within yet other embodiments of the invention, the subject is treated with radiation therapy.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. Structure of DNX-2401.

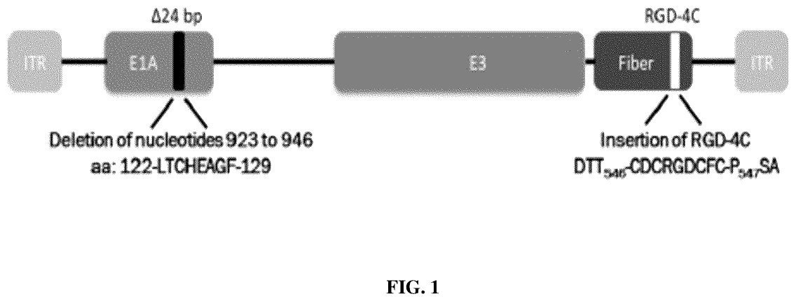

FIG. 2. Clinical visits and follow-up calendar. ¹Vital signs include Temperature, Pulse and Blood Pressure. ²Quality of Life instruments include PedQL. ³Months from DNX-2401 administration ±1 MONTH. ⁴Virology Testing may be conducted, at the discretion of the Principal Investigator, should CSF dissemination be suspected at any time. ⁵Clinical Laboratory Testing to include Hematology: white blood cell count with differential including neutrophils, lymphocytes, monocytes, eosinophils and basophils; red blood cell count; hemoglobin; hematocrit; platelets, mean platelet volume (MPV), indices MCV, MCH, MCHC, RDW. Chemistry and C-reactive protein: glucose (fasting), BUN, creatinine, sodium, potassium, chloride, calcium, total bilirubin and C-reactive protein. Coagulation: PT, PTT. ⁶Follow-up will continue every six months for survival endpoint as well as assessment and documentation of AE, concomitant medications and assessment of steroids. ⁷In the event that a study subject leaves the study early (i.e., early termination) Visit 4 assessments will be obtained with additional testing as indicated at the discretion of the Principal Investigator. Visit 4 assessments constitute the end of study visit as the final protocol-specified on-site visit. ⁸Blood samples will be obtained for potential future analysis. ⁹Can be completed up to 48 h before day of surgery. ¹⁰If the screening day and the biopsy day are within 7 days, there is no need to register again this information. ¹¹If the screening laboratory testing is performed during the same week as the biopsy day, there is no need to repeat this testing. ¹²Clinical laboratory testing for follow up will include: white blood cell count with differential including neutrophils, lymphocytes, monocytes, eosinophils and basophils; red blood cell count; hemoglobin; hematocrit; platelets, mean platelet volume (MPV), indices MCV, MCH, MCHC, RDW. Chemistry and C-reactive protein: glucose (fasting), creatinine, sodium, potassium, C-reactive protein. Coagulation: PT, PTT.

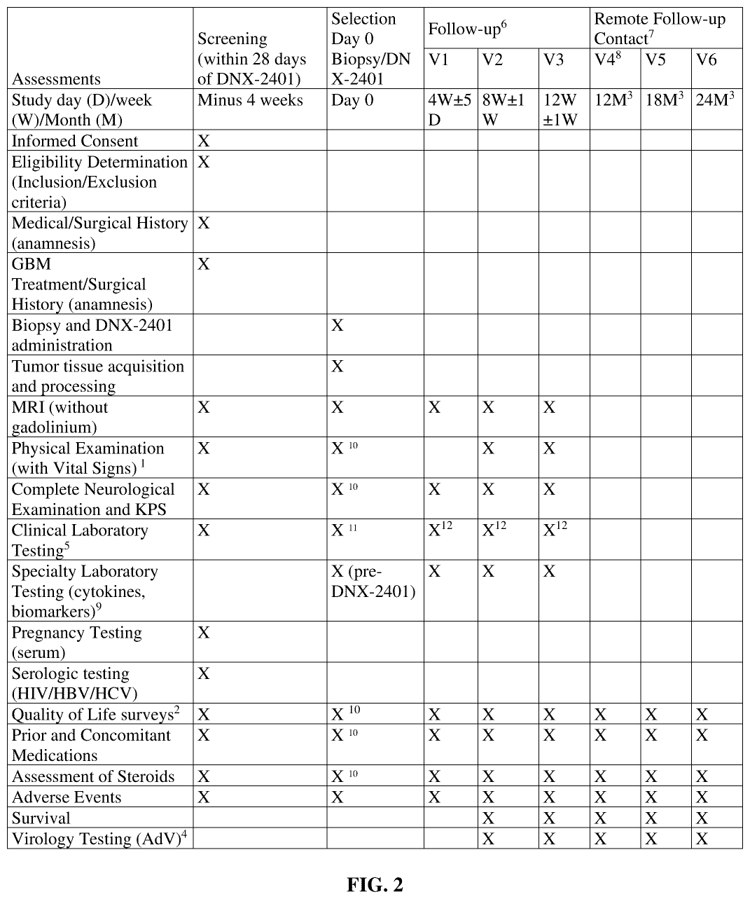

FIG. 3. Maximum percent tumor change by subject.

FIG. 4. Survival by subject.

### DESCRIPTION

The present disclosure provided methods and compositions comprising oncolytic adenoviruses for treating subjects with pediatric brain tumors. In order to further an understanding of the present invention, set forth in more detail below are sections related to: I) Replication Competent Oncolytic Viruses (including A) Adenovirus, and B. DNX-2401); II) Pediatric Brain Tumors; III) Pharmaceutical Compositions and Methods of Administration; IV) Examples; V) Various Embodiments; and VI) References.

### I. Replication Competent Oncolytic Viruses

Replication-competent oncolytic viruses according to the present disclosure include any naturally occurring (e.g., from a “field source”) or modified replication-competent oncolytic virus. The oncolytic virus, may for example, be modified to increase selectivity of the virus for cancer cells.

Particular examples of replication-competent oncolytic viruses for use in treating cancer are adenoviruses. Adenovirus (Ad) is a large (˜36 kb) DNA virus that infects humans, but which display a broad host range. Physically, adenovirus is an icosahedral virus containing a double-stranded, linear DNA genome. There are approximately 50 serotypes of human adenovirus, which are divided into six families based on molecular, immunological, and functional criteria. By adulthood, virtually every human has been infected with the more common adenovirus serotypes, the major effect being cold-like symptoms.

### A. Adenovirus

Adenoviral infection of host cells results in adenoviral DNA being maintained episomally, which reduces the potential genotoxicity associated with integrating vectors. Also, adenoviruses are structurally stable, with little if any genome rearrangement detected after extensive amplification. Adenovirus can infect most epithelial cells regardless of their cell cycle stage. Adenoviral infection is typically linked only to mild disease such as acute respiratory disease in humans.

Members of any of the 57 human adenovirus serotypes (HAdV-1 to 57) may incorporate heterologous nucleic acid encoding an immune cell stimulatory receptor agonist according to the disclosure. Human Ad5 is well characterized genetically and biochemically (GenBank M73260; AC_000008). Thus, in a preferred embodiment, the oncolytic adenovirus is a replication competent Ad5 serotype or a hybrid serotype comprising an Ad5 component. The adenovirus may be a wild type strain but is preferably genetically modified to enhance tumor selectivity, for example by attenuating the ability of the virus to replicate within normal quiescent cells without affecting the ability of the virus to replicate in tumor cells. Non-limiting examples of replication competent oncolytic adenoviruses encompassed by the present disclosure include Delta-24, Delta-24-RGD, ICOVIR-5, ICOVIR-7, ONYX-015, ColoAd1, H101 and AD5/3-D24-GMCSF. Onyx-015 is a hybrid of virus serotype Ad2 and Ad5 with deletions in the E1B-55K and E3B regions to enhance cancer selectivity. H101 is a modified version of Onyx-015. ICOVIR-5 and ICOVIR-7 comprise an Rb-binding site deletion of E1A and a replacement of the E1A promoter by an E2F promoter. ColoAd1 is a chimeric Add11p/Ad3 serotype. AD5/3-D24-GMCSF (CGTG-102) is a serotype 5/3 capsid-modified adenovirus encoding GM-CSF (the Ad5 capsid protein knob is replaced with a knob domain from serotype 3).

In one particularly preferred embodiment, the replication competent oncolytic adenovirus is Delta-24 or Delta-24-RGD. Delta-24 is described in U.S. Patent Application Publication Nos. 20030138405, and 20060147420, each of which are incorporated herein by reference. The Delta-24 adenovirus is derived from adenovirus type 5 (Ad-5) and contains a 24-base-pair deletion within the CR2 portion of the E1A gene that encompasses the area responsible for binding Rb protein (nucleotides 923-946) corresponding to amino acids 122-129 in the encoded E1A protein (Fueyo J et al., Oncogene, 19:2-12 (2000)). Delta-24-RGD further comprises an insertion of the RGD-4C sequence (which binds strongly to [symbol] v [symbol] 3 and [symbol] v [symbol] 5 integrins) into the H1 loop of the fiber knob protein (Pasqualini R. et al., Nat Biotechnol, 15:542-546 (1997)). The E1A deletion increases the selectivity of the virus for cancer cells; the RGD-4C sequence increases the infectivity of the virus in gliomas.

Oncolytic adenoviruses injected into a tumor induce cell death and release of new adenovirus progeny that, by infecting the neighbor cells, generates a treatment wave that, if not halted, may lead to the total destruction of the tumor. Significant antitumor effects of Delta-24 have been shown in cell culture systems and in malignant glioma xenograft models. Delta-24-RGD has shown surprising anti-tumor effects in Phase 1 and 2 clinical trials and is currently the subject of additional clinical trials. Although lysis of tumor cells is a major anti-cancer mechanism proposed for Delta-24-RGD oncolytic adenovirus, data from the Phase 1 clinical trial in patients with recurrent glioma and other observations indicate that the direct oncolytic effect is enhanced by the adenovirus-mediated trigger of anti-tumor immune response. Patients treated with Delta-24-RGD showed an infiltration of the tumor by immune cells that in certain cases is quite massive. Th1 and Th2 immune responses were observed that appear to correlate with optimum anti-tumor response. Aspects of the current disclosure are directed at inducing lytic activity and anti-tumor immune response for the treatment of pediatric brain cancer. Administration of oncolytic adenovirus of the disclosure leads to infection and direct lysis of tumor cells and the activation of the population of lymphocytes that recognize cancer cells with or without virus infection and accordingly provides an enhanced and prolonged antitumor effect that persists even after the virus is eradicated.

The infectious cycle of the adenovirus takes place in 2 steps: the early phase which precedes initiation of the replication of the adenoviral genome, and which permits production of the regulatory proteins and proteins involved in the replication and transcription of the viral DNA, and the late phase which leads to the synthesis of the structural proteins. The early genes are distributed in 4 regions that are dispersed in the adenoviral genome, designated E1 to E4 (E denotes “early”). The early regions comprise at least-six transcription units, each of which possesses its own promoter. The expression of the early genes is itself regulated, some genes being expressed before others. Three regions, E1, E2, and E4 are essential to replication of the virus. Thus, if an adenovirus is defective for one of these functions this protein will have to be supplied in trans, or the virus cannot replicate.

The E1 early region is located at the 5′ end of the adenoviral genome, and contains 2 viral transcription units, E1A and E1B. This region encodes proteins that participate very early in the viral cycle and are essential to the expression of almost all the other genes of the adenovirus. In particular, the E1A transcription unit codes for a protein that transactivates the transcription of the other viral genes, inducing transcription from the promoters of the E1B, E2A, E2B, E3, E4 regions and the late genes. Typically, exogenous sequences are integrated in place of all or part of the E3 region

The adenovirus enters the permissive host cell via a cell surface receptor, and it is then internalized. The viral DNA associated with certain viral proteins needed for the first steps of the replication cycle enters the nucleus of the infected cells, where transcription is initiated. Replication of the adenoviral DNA takes place in the nucleus of the infected cells and does not require cell replication. New viral particles or virions are assembled after which they are released from the infected cells, and can infect other permissive cells.

The adenovirus is an attractive delivery system. Embodiments of the disclosure can utilize a suspension cell process with average yields of 1×10¹⁶ viral particles per batch. The process can be free of or essentially free of protein, serum, and animal derived components making it suitable for a broad range of both prophylactic and therapeutic products.

Several factors favor the use of oncolytic adenoviruses for the treatment of brain tumors. First, gliomas are typically localized. Second, replication competent adenoviruses can infect and destroy cancer cells that are arrested in G0. Since gliomas invariably include non-cycling cells, this property is important. Finally, the p16-Rb pathway is abnormal in the majority of gliomas, thus making Delta-24 adenovirus particularly effective for treating these tumors, although the loss of the retinoblastoma tumor suppressor gene function has been associated with the causes of various types of tumors and is not limited to treatment of gliomas.

If an adenovirus has been mutated so that it is conditionally replicative (replication-competent under certain conditions), a helper cell may be required for viral replication. When required, helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, for example Vero cells or other monkey embryonic mesenchymal or epithelial cells. In certain aspects a helper cell line is 293. Various methods of culturing host and helper cells may be found in the art, for example Racher et al., 1995.

In certain aspects, the oncolytic adenovirus is replication-competent in cells with a mutant Rb pathway. After transfection, adenoviral plaques are isolated from the agarose-overlaid cells and the viral particles are expanded for analysis. For detailed protocols the skilled artisan is referred to Graham and Prevac, 1991.

Alternative technologies for the generation of adenovirus vectors include utilization of the bacterial artificial chromosome (BAC) system, in vivo bacterial recombination in a recA+bacterial strain utilizing two plasmids containing complementary adenoviral sequences, and the yeast artificial chromosome (YAC) system (PCT publications 95/27071 and 96/33280, which are incorporated herein by reference).

Adenovirus is easy to grow and manipulate and exhibits broad host range in vitro and in vivo. This group of viruses can be obtained in high titers (e.g., greater than 10⁹ plaque forming units (pfu) per ml), and they are highly infective. The life cycle of adenovirus does not require integration into the host cell genome.

Modifications of oncolytic adenovirus described herein may be made to improve the ability of the oncolytic adenovirus to treat cancer. Such modifications of an oncolytic adenovirus have been described by Jiang et al., (Curr Gene Ther. 2009 Oct. 9(5):422-427), see also U.S. Patent Publication No. 20060147420, each of which are incorporated herein by reference.

The absence or the presence of low levels of the coxsackievirus and adenovirus receptor (CAR) on several tumor types can limit the efficacy of the oncolytic adenovirus. Various peptide motifs may be added to the fiber knob, for instance an RGD motif (RGD sequences mimic the normal ligands of cell surface integrins), Tat motif, polylysine motif, NGR motif, CTT motif, CNGRL motif, CPRECES motif or a strept-tag motif (Rouslahti and Rajotte, 2000). A motif can be inserted into the HI loop of the adenovirus fiber protein. Modifying the capsid allows CAR independent target cell infection. This allows higher replication, more efficient infection, and increased lysis of tumor cells (Suzuki et al., 2001, incorporated herein by reference). Peptide sequences that bind specific human glioma receptors such as EGFR or uPR may also be added. Specific receptors found exclusively or preferentially on the surface of cancer cells may be used as a target for adenoviral binding and infection, such as EGFRvIII.

Representative examples of adenoviral vectors suitable for use in the present invention are described in U.S. Ser. Nos. 16/020,738, 16/997,552 and U.S. Publication Nos. 2014/0377221, 2016/0143967, 2019/0201462, 2019/0259744, all of which are incorporated by reference in their entirety.

### B. DNX-2401

DNX-2401 (tasadenoturev) is an adenovirus containing a deletion of 24 bases (bases 923-946) in the E1A gene and insertion of an integrin-binding motif (RGD) in the H1 loop of the fiber. It is an oncolytic virus that infects and replicates in cells with a functionally inactive Rb pathway. It may replicate in dividing cells that have transient decrease in Rb. Cell killing is thought to occur through oncolysis that results in release of infectious viral particles within the tumor.

DNX-2401 has demonstrated a potent anti-tumor mechanism of action by (1) replicating in human tumors (2) eliciting tumor necrosis and (3) triggering an immune response. The first Phase 1 trial performed with DNX-2401 in recurrent high-grade glioma demonstrated both biological and clinical activities of the virus (Lang et al., 2018). Replication of DNX-2401 was evident in 6 of 11 tumors resected 14 days after a single intratumoral injection of 1×10⁷-3×10⁸ vp DNX-2401. Radiographic signs of inflammation, histopathologic evidence of tumor infiltration by CD8+, T-bet+ cells, and TIM-3 downregulation after treatment were also observed. Analyses of patient-derived cell lines for damage-(or danger-) associated molecular patterns (DAMP) revealed induction of immunogenic cell death in tumor cells after DNX-2401 administration. Treatment with DNX-2401 resulted in dramatic responses with long-term survival in recurrent high-grade gliomas that are probably due to direct oncolytic effects of the virus followed by elicitation of an immune-mediated anti-tumor response.

### II. Pediatric Brain Tumors

As noted above, the compositions and methods of the present invention can be used to treat a variety of pediatric brain tumors, including for example, gliomas. As utilized herein, “pediatric” patients range in age from 1 to 18 years old. However, within certain embodiments of the invention the therapies are particularly suitable to patients from with the worst prognosis (e.g., patients from 3 to 10 years of age. Representative examples of pediatric brain cancers include gliomas such as Diffuse Intrinsic Pontine Glioma and high-grade gliomas, Atypical Teratoid/Rhabdoid Tumors (“AT/RT”), and a Primitive Neuroectodermal Tumors (“CNS-PNET”).

Particularly preferred tumors to be treated are Diffuse Intrinsic Pontine Gliomas or DIPG, a form of brainstem glioma that is one of the most lethal pediatric tumors.

Tumor cells of a developing DIPG gradually compress crucial nuclei and tracts within the pons. As the tumor enlarges, it causes symptoms due to impaired function of neurons arising in or running through the pons that carry information to and from the cerebellum, cerebral cortex, spinal cord, and cranial nerves. The most common symptoms are a triad of 1) cerebellar deficits such as impaired balance and coordination, 2) long tract impairment causing weakness or sensory loss of the extremities or trunk, and 3) paresis of cranial nerves VI and VII, which facilitate outward movement of the eye and face, respectively. Headaches, altered level of consciousness and other cranial nerve deficits due to obstruction of cerebral spinal fluid flow due to tumor impingement on the ventricular system of the brain, are also not uncommon.

Once a clinician suspects a brainstem abnormality, the radiologic study of choice is Magnetic Resonance Imaging (MRI). DIPG has a peak incidence of 6-9 years and typically exhibits rapid clinical onset and progression with 90% mortality rate within 18 months of presentation, with less than 2% surviving after 5 years. The median overall survival with standard of care radiation therapy (RT) is approximately 8-10 months. Overall survival at 12 months (OS12) is approximately 35% and survival at 2 years and 5 years is less than 10% and 1%, respectively.

As discussed above, there currently are no effective treatments for DIPG; these tumors are not amenable to surgical resection due to the diffuse nature and location of DIPG, and diagnosis is based on characteristic appearance on MR Imaging and/or molecular profiling. Mutation in histone H3 is present in approximately 90% of subjects with DIPG, typically occurring in variants H3.3 (60%) or H3.1 (30%)(Cohen 2017). Wildtype histone H3 or mutations in H3.3 are considered prognostic indicators for poor survival, compared to H3.1 mutations. A mutation in p53 is also considered a poor prognostic indicator and is associated with resistance to radiation therapy (Werbrouck 2019).

Conventional radiation therapy (RT) is typically used for palliation, providing a transient improvement in neurologic function and progression free survival benefit up to one year with minimal side effects. However, median onset of disease progression following RT is less than 6 months, with no improvement in overall survival. Prolonged survival up to 24 months has been observed in less than 10% of patients. Clinical trials to date have not led to advancement in the treatment paradigm of DIPG.

Within certain embodiments of the invention, the tumor of a subject with a pediatric brain tumor is radiographically confirmed. Within further embodiments of the invention, the tumor is analyzed on a molecular basis for mutations. Within certain embodiments of the invention, the subject has a H3.3K27M, H3.1K27M, H3.2K27M, wild type H3, H3.3G34RV, and/or p53 mutation (see generally, Mackay et al, 2017 Cancer Cell 32:520-537, Oct. 9, 2017, which is incorporated by reference in its entirety). As set forth in more detail below, significant therapeutic effects have been seen in subjects that have been treated with the oncolytic adenoviral vectors (e.g., DNX 2401) provided herein, along with radiation therapy.

### III. Pharmaceutical Compositions and Methods of Administration

The present disclosure also provides a pharmaceutical composition comprising any composition of the present disclosure, and a pharmaceutically acceptable carrier.

The present disclosure also provides a method of treating DIPG in a pediatric subject, comprising administering to a subject a composition of the present disclosure.

According to the present disclosure, an oncolytic adenovirus is administered to a subject to induce an immune response for therapeutic or prophylatic purposes. Thus, in certain embodiments, the expression construct is formulated in a composition that is suitable for this purpose. The phrases “pharmaceutically” or “pharmacologically acceptable” refer to compositions that do not produce adverse, allergic, or other untoward reactions when administered to an animal or a human. As used herein, “pharmaceutically acceptable carrier” includes any and all solvents, carriers, dispersion media, coatings, antibacterial and antifungal agents, isotonic and absorption delaying agents, and the like. The use of such media and agents for pharmaceutically active substances is well known in the art. Except insofar as any conventional media or agent is incompatible with the expression constructs of the present disclosure, its use in therapeutic compositions is contemplated. Supplementary active ingredients also can be incorporated into the compositions.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. If needed, various antibacterial an antifungal agents can be used, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating compounds in the required amount in the appropriate solvent with various of the other ingredients enumerated above, as required, followed by filter sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically or prophylactically effective. For parenteral administration in an aqueous solution, the solution should be suitably buffered if necessary and the liquid diluent first rendered isotonic with sufficient saline or glucose. These particular aqueous solutions are especially suitable for intravascular and intratumoral administration. In this connection, sterile aqueous media, which can be employed will be known to those of skill in the art in light of the present disclosure.

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by the FDA.

Dosage—An effective amount of the therapeutic or preventive virus is determined based on the intended goal, for example stimulation of an immune response against a tumor. Those of skill in the art are well aware of how to apply gene delivery in vivo and ex vivo situations. For viral vectors, one generally will prepare a viral vector stock. Depending on the kind of virus and the titer attainable, one will deliver at least about, at most about, or about 1×10⁴, 1×10⁵, 1×10⁶, 1×10⁷, 1×10⁸, 1×10⁹, 1×10¹⁰, 1×10¹¹ or 1×10¹² infectious particles, or any value or range there between, to a subject. In other aspects, adenoviruses according to the disclosure may be administered in a single administration or multiple administrations. The virus may be administered at dosage of 1×10⁵ plaque forming units (PFU), 5×10⁵ PFU, at least 1×10⁶ PFU, 5×10⁶ or about 5×10⁶ PFU, 1×10⁷, at least 1×10⁷ PFU, 1×10⁸ or about 1×10⁸ PFU, at least 1×10⁸ PFU, about or at least 5×10⁸ PFU, 1×10⁹ or at least 1×10⁹ PFU, 5×10⁹ or at least 5×10⁹ PFU, 1×10¹⁰ PFU or at least 1×10¹⁰ PFU, 5×10¹⁰ or at least 5×10¹⁰ PFU, 1×10¹¹ or at least 1×10¹¹, 1×10¹² or at least 1×10¹², 1×10¹³ or at least 1×10¹³ PFU. For example, the virus may be administered at a dosage of between about 10⁷-10¹³ PFU, between about 10⁸-10¹³ PFU, between about 10⁹-10¹² PFU, or between about 10⁸-10¹² PFU.

Replication-competent oncolytic viruses according to the disclosure may be administered locally or systemically. For example, without limitation, oncolytic viruses according to the disclosure can be administered intravascularly (intraarterially or intravenously), intratumorally, intramuscularly, intradermally, intraperitoneally, subcutaneously, orally, parenterally, intranasally, intratracheally, percutaneously, intraspinally, ocularly, or intracranially. In preferred embodiments, an adenovirus of the disclosure is administered intravascularly or intratumorally.

Within other embodiments of the invention the oncolytic viruses can be administered via a direct administration into the brain, e.g., by a fine catheter or cannula (e.g., a neuroventricular cannula). Within certain embodiments of the invention, replication competent oncolytic viruses can be delivered under MRI intra-procedural guidance. Preferably, intratumoral administration into the brain is accomplished without significant reflux or back flow by using a cannula or catheter with a step feature such as the SmartFlow cannula (Clearpoint Neuro Inc.), the Brainlab Flexible catheter (Brainlab AG), or Alcyone MEMS cannula (AMC, Alycone Lifesciences Inc.). Representative examples of such devices are described in U.S. Pat. Nos. 8,992,458, 9,919,129, 10,137,244, 10,434,251, 10,441,770 and 10,806,396, all of which are incorporated by reference in their entirety.

Replication-competent oncolytic viruses according to the disclosure may also be administered in a cellular carrier. In this respect, neuronal and mesenchymal stem cells have high migratory potential yet remain confined to tumor tissue. A subpopulation of adult mesenchymal cells (bone marrow derived tumor infiltrating cells or BM-TICs) has been shown, following injection into gliomas, to infiltrate the entire tumor. Thus, oncolytic viruses according to the disclosure can be administered in a virus-producing neuronal or mesenchymal stem cell (e.g. BM-TIC) carrier (e.g. by injection of the carrier cell into the tumor)

The quantity to be administered, both according to number of treatments and dose, depends on the subject to be treated, the state of the subject and the protection desired. Precise amounts of the therapeutic composition also depend on the judgment of the practitioner and are peculiar to each individual.

Within certain preferred embodiments of the invention, in addition to an oncolytic adenoviral vector as described herein, the patient is administered a therapeutic agent that produces or stimulates an immune response (e.g., it upregulates or activates the cellular immune system or, is an antagonist of a suppressor of cellular immunity (i.e., antagonist of cellular immune-suppression). Antagonists of cellular immune-suppression are agents that act on cells or molecules that suppress the cellular immune system. Antagonists of cellular immune-suppression include cytotoxic T-lymphocyte antigen 4 (CTLA-4; also known as CD152) antagonists such as Ipilimumab (also known as Yervoy™, MDX-010 or MDX-101; a humanized monoclonal antibody against CTLA-4 developed by Bristol-Myers Squibb) and Tremelimumab (formerly ticilimumab, CP-675,206; a humanized monoclonal antibody against CTLA-4 MedImmune/AstraZeneca); PD-1/PD-L1-receptor antagonists such as MDX-1106 (an α-PD-1 humanized monoclonal antibody, Bristol-Myers Squibb); MK-3475 (pembrolizumab) (an α-PD-1 humanized monoclonal antibody, Merck); AMP-224 (Fc-PD-1 fusion protein that blocks interaction between PD-1 and ligands B7-DC and B7-H1; Glaxo Smith Kline); Pidilizumab (also known as CT-011; a humanized monoclonal antibody against αPD-1, Chirotech); MDX-1105 (an α-PD-L1 humanized monoclonal antibody, Bristol-Myers Squibb); antibodies that specifically bind to B7-H3 such as MGA271 (an α-B7-H3 humanized monoclonal antibody, Microgenics) or other antibodies as described in US Application Publication Number 2012-0294796, the contents of which are incorporated herein by reference; or indoleamine-2,3-dioxygenase (IDO) inhibitors such as D-1-methyl-tryptophan (Lunate) and other compounds described in U.S. Pat. No. 7,799,776, the contents of which are incorporated herein by reference.

Within yet further embodiments of the invention, the subject is treated with an immune cell stimulatory receptor agonist (e.g., a PD-1/PD/L1 receptor antagonist such as for example, MDC-1106, MK-3475 (pembrolizumab), AMP-224, Pidilizumab and MDX-1105.

Within yet other embodiments of the invention, the subject is treated with conventional or hyperfractionated radiation therapy.

### IV. Examples

The following examples as well as the figures are included to demonstrate preferred embodiments of the disclosure. It should be appreciated by those of skill in the art that the techniques disclosed in the examples or figures represent techniques discovered by the inventors to function well in the practice of the disclosure and thus can be considered to constitute preferred modes for its practice. However, those of skill in the art should, in light of the disclosure, appreciate that many changes can be made in the specific embodiments which are disclosed and still obtain a like or similar result without departing from the spirit and scope of the disclosure.

### Example 1—Hypothesis and Study Objectives

Some oncolytic viruses have proved efficacy in animals and have been used in humans, but these agents has shown very little efficacy in humans until now. In this trial, the inventors propose to use the oncolytic adenovirus DNX-2401. The results achieved with DNX-2401 in animal models have been impressive.

This virus is engineered with two genetic modifications that allows for selective antitumoral effect. The first change is a deletion of 24 nucleotides in the E1A protein (Delta-24), which allows the virus to replicate in cells with an abnormal RB pathway. Inactivation of this pathway is a common trait in nearly all tumors.

The second modification is the addition of a peptide in the adenovirus fiber (RGD), the peptide makes the virus able to attach and infect the cell through integrins, which are a membrane receptor very abundant in glioblastoma cells, instead of the normal affinity for coxsackie-adenovirus receptors (CARs) which are scarce in GBM. This modified DNX-2401 adenovirus has shown great efficacy in animal models.

Because the diffuse and infiltrative characteristic of DIPGs, a targeted therapy, such as an oncolytic virus, could provide a better outcome for children affected by this devastating tumor and constitute the future treatment of these tumors. However, in order to become a real therapeutic alternative for these tumors, more information about them is still needed. Recent technological advances have made rapid profiling of tumor DNA and RNA possible, revealing differences in gene expression that could help explain tumor formation and be used to develop drug targets. Scientists have also been able to develop reliable transgenic animal models of DIPG, which have facilitated characterization of tumor cell behavior and also the investigation of therapeutic targets.

If DNX-2401 works as it is expected, it will kill the tumor cells with no damage of the healthy tissue. Because the virus is killing the tumor cells, it could result in brain edema around the tumor along the virus effect. If intense, edema could produce neurological symptoms; however, it is important to note edema has not been seen with this virus in all the trails performed until now.

The primary study objectives and endpoints are to determine the safety, tolerability and toxicity of DNX-2401 injected in the cerebellar peduncle in pediatric subjects with DIPG. The trial will look for hematologic and neurologic toxicity (NCI-CTCAE v 4.03). The secondary study objective are to determine Overall Survival at 12 months (OS12), complete/partial response by MRI, immune response induced by DNX-2401, to measure quality of life (QoL) baseline assessment and any changes over time, and to collect tumor and blood samples for future molecular and immune studies.

### Example 2—Study Design and Procedure

Design summary. The proposed study is a Phase I, unicentric, and uncontrolled trial with a 3+3 design. Intratumoral injection of DNX-2401 through cerebellar peduncle will be followed by standard therapy of radiotherapy and chemotherapy in 4 to 6 weeks.

Screening and selection period. Patients will be screened in the clinic by the investigators of the trial. Screening will take place within 28 days of selection and administration of DNX-2401. Screening will be preceded by a presentation of the complete information about the clinical study to the parents and the subject by the investigator followed by signature of the informed consent form. Enrollment to the trial will be possible only after the following screening tests are performed and results are found to be in compliance with inclusion and exclusion criteria for enrollment:
Recent clinical and radiological diagnosis of DIPG.
Physical Examination, including vital signs and weight
Neurological Examination (including Lansky/Karnofsky Performance Status (KPS))
Quality of Life instruments (PedsQL™)⁷
Clinical Laboratory Tests including hematology, chemistry (with C-reactive protein) and coagulation (at screen, or recent standard of care, with PI approval)
Pregnancy testing (serum) (at screen, or recent standard of care, with PI approval)
Serology Testing for HIV and HBV/HCV (at screen, or recent standard of care, with PI approval)
MRI with and without gadolinium (at screen, or recent standard of care, with PI approval)
Current medication listing

In order to prevent subjects becoming lost to follow-up, the subject will be asked to supply information for 1-2 people who do not reside with her/him and who might be contacted to provide survival information for follow-up should the subject be unreachable at the primary residential contact provided.

Treatment Period. DNX-2401 will be injected immediately after the biopsy. The injection will be intratumoral through the biopsy tract in the cerebellar peduncle. The most effective dose in previous trials has been 5×10¹⁰ (hereinafter referred to as dose D2). Because the virus will be injected in the pons, we have decided to start with a lower dose: 1×10¹⁰ (hereinafter referred to as dose D1) to check there is not brain edema, before injecting the final dose (D2). The virus must be injected in the brain, when the virus is injected anywhere else, this virus cannot reach the brain.

A cohort of 3 patients will receive the D1 dose (1×10¹⁰); if there is not toxicity grade III-IV related to the virus during the first 2 months, the next patients will receive D2 dose (5×10¹⁰). If there is toxicity in 2 or 3 patients, the number of viral particles will be reduced 1×10⁹ (hereinafter referred to as dose D0). If there is toxicity in 1 patient, another cohort of 3 patients will be injected with D1 dose (1×10¹⁰). If there is toxicity in 1 patient, there will not be a dose escalation, the dose will be decreased and all the patients will receive D0 (1×10⁹).

Tumor tissue obtained at biopsy or resection will be archived for tumor sequencing and tumor banking in the tumor bank of our hospital. Additional blood testing for future analysis will be obtained.

Administration of DNX-2401 should be interrupted if a subject develops symptoms of decreased cerebral function or diminished cardiovascular perfusion or if there are signs of allergic reaction or anaphylaxis. DNX-2401 administration will also be interrupted for any adverse event that, in the Investigator's opinion, warrants interruption. In addition, if it appears that ventricular penetration has occurred, the administration procedure will be aborted and no further DNX-2401 will be administered. Suspected dissemination of DNX-2401 into and throughout the ventricular system/cerebrospinal fluid will be investigated by virology testing (AdV) and further follow-up.

In order to improve the information in the MRI performed after closure, gadolinium will be infused through the Alcyone cannula.

Procedure overview for reference:
Dilute the Gadolinium with saline to 20 mM
Prime diluted Gadolinium into one of the independent lumens of the AMC
Prime the DNX-2401 into the second independent lumen of the AMC
Insert AMC on planned trajectory for optimal biodistribution
First infuse 150-250 μL of Gadolinium
Then infuse the 1 ml of the DNX-2401
Remove AMC and close the patient

After completion of DNX-2401 administration and wound closure an MRI without gadolinium will be performed, under the same general anesthesia. This MRI will be the baseline scan for the study.

The subject will be given a study wallet identification card at hospital discharge to provide to outside caregivers and medical personnel the investigator's contact information in case of an emergency.

The radiation therapy will start at radiotherapist discretion, 2-6 weeks after biopsy and virus injection, as well as chemotherapy. Each patient will follow the treatment indicated by his/her pediatric oncologists, and the radiological studies and clinical visits will be done in their reference hospital. The images will be sent to our investigators, and MRIs could be performed in Clinica Universidad de Navarra if the investigator considers it necessary.

Follow-up period. Clinical Follow-up. After inclusion in the study clinical visits will be made every four weeks, during the first 3 months. After the end of the 3 months, the follow up could be done by phone, sending the images and blood tests by mail, or in-person if the patient prefers to be treated in our hospital. The National Cancer Institute-Common Terminology Criteria (NCI-CTCAE) version 4.03 will measure treatment tolerance through severity grading of Adverse Events.

### Visit Schedule:

Visit 0 (Treatment day). At this visit the assessments will include:
Physical Examination, including vital signs, weight, and height
Neurological Examination (Lansky scale)
Quality of Life (PedsQL™)
Hematology, Chemistry (with C-reactive protein) and Coagulation
Additional specialty laboratory blood testing for future analysis
Assessment and documentation of adverse events, concomitant medications
Assessment of steroids
Biopsy and DNX-2401 administration
Tumor tissue acquisition and processing

Visit 1 (28±5 days following the Injection of DNX-2401). At this visit the following assessments will be performed:
Physical Examination, including vital signs, weight, and height
Neurological Examination (Lansky scale)
Quality of Life (PedsQL™)
Hematology, Chemistry (with C-reactive protein) and Coagulation
Additional specialty laboratory blood testing for future analysis
Assessment and documentation of adverse events, concomitant medications
Assessment of steroids

Visit 2 (8±1 weeks post DNX-2401). At this visit the following assessments will be performed:
Physical Examination, including vital signs
Neurological Examination
Quality of Life (PedsQL™)
Hematology, Chemistry and Coagulation
Additional blood testing for future analysis.
MRI with tumor response assessment
Assessment and documentation of adverse events, concomitant medications
Assessment of steroids

Visit 3 (12±1 weeks post DNX-2401). At this visit the following assessments will be performed:
Physical Examination, including vital signs
Neurological Examination and KPS
Quality of Life (PedsQL™)
Hematology, Chemistry (including C-reactive protein) and Coagulation
Additional blood testing for future analysis
MRI with tumor response assessment
Assessment and documentation of adverse events, concomitant medications
Assessment of steroids
Visit 3 assessments constitute the end of study visit as the final protocol-specified on-site clinic visit.

Unscheduled Visit (To be determined). At this visit the following assessments will be performed:
At any time during the study, the patient may be seen in clinic for a visit outside of the protocol-specified visit schedule. Selected tests as required may be performed at the discretion of the Principal Investigator.
Virology (AdV) testing may be conducted, at the discretion of the Principal Investigator, should CSF dissemination be suspected at any time.

Remote Follow-up Contact (3 months±2 weeks post DNX-2401 and every six months±1 MONTH after Month 3):
Clinical follow-up by telephone or clinic visit
Survival
Assessment and documentation of adverse events, concomitant medications
Assessment of steroids

After the last clinical visit of the trial, patients will follow standard care under supervision of his or her pediatric oncologist; the investigator will collect basic information to determine secondary end point Overall Survival.

If the subject is able to come to the clinic, these visits will include complete physical exam, complete neurological exam, and quality of life assessment. The National Cancer Institute-Common Terminology Criteria for Adverse Events will measure treatment tolerance (NCI-CTCAE, version 4.03).

The frequency of clinical visits could increase if it would be deemed necessary because of clinical situation, incapacity or demands of the patient.

At tumor progression, the procedures of the study will end, and the patient will follow the treatment at the discretion of his/her pediatric oncologist, however, basic information about clinical status will be collected, at least by telephone, until death or at least 2 years from virus injection. This basic information will include at least if patient is alive or dead, and an estimation of quality of life.

If there is no progression, basic information about the situation of the patient will be collected, at least by telephone, until death or until five years. This basic information will include at least if patient is alive or dead, treatment followed and an estimation of quality of life. The Principal Investigator may decide to conduct final analysis of study data at any time, even while some subjects may remain in long-term follow-up. This decision may also take place after all study subjects have died or been followed for more than one year. The timing of data analysis is the prerogative of the Principal Investigator, regardless of continuing long-term follow-up of study subjects.

Laboratory test follow-up. Basic blood test would be done before inclusion and in visits 1 to 3. These tests will include, but may not be limited to:
Hematology: white blood cell count with differential including neutrophils, lymphocytes, monocytes, eosinophils and basophils; red blood cell count; hemoglobin; hematocrit; platelets, mean platelet volume (MPV), erythrocyte sedimentation rate (ESR) and indices MCV, MCH, MCHC, RDW.
Chemistry and C-reactive protein: glucose (fasting), creatinine, sodium, potassium, chloride, calcium- and C-reactive protein.
Coagulation: PT, PTT.

Specialty lab tests: Blood samples will be collected before and after DNX-2401 injection for the analysis of tumor markers and future analysis. Following permission request from the patient, blood samples may be retained and archived for future analysis. If available, tumor tissue may be tested for gene sequencing. Tumor tissue may be archived for future analysis, if available.

### Radiology Follow-Up:

MRI will be used to follow tumor response. As long as similar sequences are used both 1.5T and 3T can be used indistinctly. The MRI protocol will include the following sequences:
T2 FLAIR 3D sag.
T1 GE 3D axial without intravenous contrast and 1×1×1 mm voxel size, T2 TSE axial 4.0 mm slice thickness, SWI axial 2.0 mm slice thickness, DIFFUSION EPI 2D axial b0 b1000 ADC 4.0 mm slice thickness, and T2 FLAIR 3D SPACE sagittal 1 mm slice thickness. OPTIONAL: T1 GE 3D axial with fat saturation 1×1×1 mm voxel size after a single dose of intravenous contrast administration (Gadovist 0.1 ml/kg) for enhancing tumors, T2* PERFUSION EPI 4 mm slice thickness, DTI EPI 2D axial 4.0 mm slice thickness.

MRI will be performed at the following time points:
At screening prior to selection to assist in establishing eligibility.
Immediately following post DNX-2401 administration to provide the baseline.
At visit 2 (approximately 12 weeks after DNX-2402 administration. After radiotherapy).
At visit 3 (the end of the visits of the trial).

The Principal Investigator may require an MRI at any time during the study in order to investigate possible tumor recurrence or to confirm response. Response should be confirmed at 4 weeks (not less than 28 days) from initial determination.

RAPNO criteria (Cooney, 2020)⁹ will be used to evaluate response to therapy. In the event that pseudoprogression is suspected due to image changes within the first two months after virus injection, the MRI would include advanced sequences, at radiologist criteria. After the last specified MRI, study procedures will finish, and the patient will continue standard of care after the criteria of his or her treating pediatric oncologist and be followed for survival endpoint. An MRI may be ordered at any time, per the discretion of the Principal Investigator, to investigate clinical signs suggestive of a tumor recurrence or to confirm response.

In the event of neurological worsening that can be suggestive of progression upon the criteria of the investigator, the schedule of the next MRI will be changed, and the study could be performed within one week for the worsening if the pediatric oncologist is not requesting an MRI.

The radiological procedures of the study will finish 12 weeks after DNX-2401 injection. After that, the patient will follow standard follow-up on the criteria of his/her treating pediatric oncologist; the investigators will keep collecting basic clinical and radiological information, until death or 2 years from inclusion.

### Example 3—Study Population

Inclusion criteria. Patients must meet the following criteria to be included in the trial:
1. Informed consent of patient or parents.
2. Patient must be, in the investigator opinion, able to comply with all the protocol procedures.
3. Age 1-18 years
4. Negative pregnant blood test in case of fertile women (A woman is considered of childbearing potential (WOCBP), i.e., fertile, following menarche and unless permanently sterile. Permanent sterilization methods include hysterectomy, bilateral salpingectomy and bilateral oophorectomy.
5. Patient newly diagnosed of DIPG in MRI. The radiologist will confirm that the MRI is diagnostic of DIPG following the radiological criteria: defined as a T1-hypo (or iso) intense and T2-hyperintense tumor involving at least 50% of the pons.
6. Lansky/Karnofsky Performance Status ≥70 before inclusion
7. Lesion considered by the investigator to be accessible for stereotactic biopsy. Lesion location will allow injection without entrance of virus in the ventricular system.
8. No previous treatment for DIPG

### Exclusion Criteria.

Patients with any of the following exclusion criteria could not be included in the trial:
1. Severe infections or intercurrent medical conditions including, but not limited to, severe renal, hepatic, heart or bone marrow failure, that, on investigator's criteria, do not allow the inclusion. Patients must be afebrile at baseline [i.e., <38 degrees (C^{o})].
2. Investigational medication in the previous 30 days.
3. Subjects with immunodeficiency, autoimmune conditions or active hepatitis.
4. Any medical or psychological condition that might interfere with the subject's ability to participate if older than 16 years or parents ability when younger than 16, or give informed consent or would compromise the patient's ability to tolerate therapy or any disease that will obscure toxicity or dangerously alter drug metabolism.
5. Tumor with multiple locations or doubt in MRI of a DIPG.
6. Pregnant or breast-feeding females will be excluded, due to the risk for the fetal development of a recombinant virus containing genes related to cellular growth and differentiation.
7. Severe bone marrow hypoplasia.
8. Neutrophils <1×10⁹/L
9. Thrombocytes≤100×10⁹/L
10. Hemoglobin <9 g/dl
11. Patients with Li-Fraumini Syndrome or with a known germ line deficit in the retinoblastoma gene or its related pathways.
12. Vaccinations of any kind within 30 days prior to DNX-2401 administration.
13. Transfusions or medications (G-CSF) to treat pancytopenia or other hematological conditions within 28 days of baseline.

Screening and selection procedure. Entry in the study will be offered to every patient evaluated in the center that fulfill all the inclusion criteria and do not have any of the exclusion criteria. Patients will be screened for the study when a DIPG is diagnosed by MRI, or even after a biopsy, with not previous treatment of radiotherapy or chemotherapy. The patient will be included in the trial after the inform consent is signed. The study aims to enroll 12 patients.

Withdrawal criteria. The investigator could decide to withdraw a patient from the study, due to any adverse event, depending on the severity and the relationship of the adverse event to the experimental therapy. All adverse events will be followed and documented at least 12 weeks (84 days) from virus injection. Related events will be followed until Grade 1 or stable. Radiology and clinical follow-up will continue until 6 months, if progression is not diagnosed before. Patient can withdraw from the study in any moment without the need for an explanation. If possible, the investigator will try to complete the clinical data in that moment. The patients withdrawn will not be substituted, because they will be followed for the OS and the tissue samples will be analyzed.

Duration of participation of each patient. Each patient will receive the virus injection once, before any other tumor therapy and will be in clinical and radiological follow-up during 12 weeks. After this date, patients will be asked to notify any radiological change or therapeutic change until two years. Patients who may be alive at 24 months post DNX-2401 will continue to be followed for survival or five years. The investigator team will contact each patient at 6 months, 12 months, 18 months and 24 months, by telephone or mail to update data about progression free survival and overall survival.

### Example 4—Description of Study Therapy

Trial drug. DNX-2401 is an adenovirus containing a deletion of 24 bases (bases 923-946) in the E1A gene and insertion of an integrin-binding motif (RGD) in the H1 loop of the fiber. DNX-2401 is formulated in 20 mM Tris, 25 mM NaCl, 2.5% (w/v) Glycerol, pH 8.0 (GST buffer). Lot P817003 was vialed at a concentration of 2.0×10¹¹ vp/mL.

DNX-2401 is an oncolytic virus that infects and replicates in cells with a functionally inactive Rb pathway. It may replicate in dividing cells that have transient decrease in Rb. Cell killing is thought to occur through oncolysis that results in release of infectious viral particles within the tumor.

Formulation and storage. Formulation: The final vialed product, Lot P817003 is DNX-2401 formulated in 20 mM Tris, 25 mM NaCl, 2.5% (w/v) Glycerol, pH 8.0 (GST buffer). Lot P817003 was vialed at a concentration of 2.0×1011 vp/mL. Appearance is clear to translucent, colorless liquid with no evidence of particulate matter.

Final vialed biological product DNX-2401 Lot P817003 is contained in 2.0 mL clear glass vials manufactured by Schott with gray butyl stoppers and sterile red flip-off button crimps manufactured by West. Vials are filled to 0.45 ml to permit extraction of 0.2 mL. Frozen glass vials (585) were boxed and transferred to Quality Control at Lonza Houston, Inc. for controlled storage at ≤−60° C. on Jan. 18, 2013.

Dosing. Total dose will be D0=1×10⁹, D1=1×10¹⁰ or D2=5×10¹⁰ viral particles (vp) suspended in 1 ml for all cases. Virus will be kept in an ≤−70 C freezer, in the vial in which it was provided. Vial concentration is 2.0×10¹¹ vp/mL. One dilution will be required to produce the total dose for the study of 5×10¹⁰ vp/mL in 1.0 mL as the dose to be delivered. However, it may be necessary to prepare enough DNX-2401 in order to fill the dead space in delivery equipment in order to ensure delivery of the complete dose. The Alcyone cannula to be used in the trial has a dead volume of 50 μL, and the tubing has a dead volume of 500 μL. That is why a total volume of 1.6 mL will be loaded in the syringe (see below).

The virus will be diluted and prepared in the Pharmacy of the hospital and send to the Operating Room in a syringe, ready for injection. See below the procedure for each dose: D0, D1 and D2.

| | |
|---|---|
| VIRUS VIAL CONCENTRATION DNX-2401 | 2.0 × 10¹¹ vp/mL. |
| FILL VOLUME OF THE VIAL | 0.4 mL |
| EXTRACTABLE VOLUME | 0.2 mL |
| DEAD SPACE OF THE CANNULA, EXTENSION | 500 μL |
| TUBING AND ADDITIONAL FLUSH | |
| DILUTIONS | |
| D1: 1 × 10¹⁰ viral particles | Load 0.2 mL of DNX-2401 vial and add 3.8 |
| (vp) suspended in 1 mL | mL of 0.9% Sodium Chloride |
| | (concentration: 4 × 10¹⁰ vp in 4 mL final |
| | volume). |
| | Gently mix to homogenize the solution in |
| | the vial (without overturning). |
| | Load 1.6 mL of the “Diluted DNX-2401” |
| | solution in a Luer-lock BD Plastipak syringe |
| | of 5 mL. |
| | Cover with red Luer cap. |
| | Introduce in sterile labeled bag (label with |
| | clinical history number, Date, and final |
| | volume). |
| | Introduce in a second non-sterile bag. |
| D2: 5 × 10¹⁰ viral particles (vp) | Load 1.2 ml of saline in a syringe with an |
| suspended in 1 mL | appropriate gauge needle and put it in the |
| | vial of DNX-2401 (final volume: 1.6 mL) |
| | Gently mix to homogenize the solution in |
| | the vial (without overturning). |
| | Load 1.6 mL of the “Diluted DNX-2401” |
| | solution in a Luer-lock BD Plastipak syringe |
| | of 5 mL. |
| | Cover with red Luer cap. |
| | Introduce in sterile labeled bag (label with |
| | clinical history number, Date, and final |
| | volume). |
| | Introduce in a second non-sterile bag. |
| D0: 1 × 10⁹ viral particles (vp) | Load 0.2 mL of DNX-2401 vial and add 3.8 |
| suspended in 1 mL | mL of 0.9% Sodium Chloride |
| | (concentration: 4 × 10¹⁰ vp in 4 mL final |
| | volume). Labelled as “A Solution.” |
| | Gently mix to homogenize the solution in |
| | the vial (without overturning). |
| | Load 1 mL of the “Dilution A” and add 9 |
| | mL of Sodium Chloride 0.9% |
| | (concentration: 1 × 10¹⁰ vp in 10 mL final |
| | volume: 1 × 10⁹ vp/mL). Labelled as “B |
| | solution.” |
| | Gently mix to homogenize the solution in |
| | the vial (without overturning). |
| | Load 1.6 mL of the “B solution” in a Luer- |
| | lock BD Plastipak syringe of 5 mL. |
| | Cover with red Luer cap. |
| | Introduce in sterile labeled bag (label with |
| | clinical history number, Date, and final |
| | volume). |
| | Introduce in a second non-sterile bag. |

Drug accountability. The drug will be received at the Pharmacy department, following local regulation, with receipt acknowledged by signing the receipt confirmation list that will be filed at site pharmacy file. Containers received will be detailed at Pharmacy internal database that will be available during Site monitoring visit. All the virus vials received in the pharmacy in each shipment will be stored in the following way:

Virus will be kept in a −80° C. freezer. Refrigerator temperature will be taken graphically continuously. Temperature logs can be check at every monitoring visit and in case of inspection.

The following information is registered in site internal accountability electronic logs for clinical trials: investigational product, number of assigned vials of IMP used, batch and expiration date of such vials, dose, patient's identification, quantity vials dispensed, person responsible for the accountability register.

Site accountability electronic logs will be available for monitoring visits.

Used Vials/containers will not be available for checking in monitoring visits for safety reasons, according to internal procedures.

All materials that have been in contact with DNX-2401 will be considered as infectious biohazards.

Unused IMP will only be destroyed after Monitor approval. Approval drug destruction at site will be filed at pharmacy folder as well as the Certification of destruction. Hereby it is certified that the Site has a destruction policy in place.

Delegated pharmacy staff will be responsible for medication destruction at site and accountability of study drug.

Handling and disposal procedures. DNX-2401 handling and disposal will follow Institutional standards for Biosafety Level 2 agents. In addition, standard chemotherapy preparation precautions (gown, gloves, mask, and glasses) and sterile technique while preparing the required dose of DNX-2401 will be followed.

Full-strength decontaminant (e.g., Dispatch or equivalent) will be in the laminar flow biological safety cabinet in a plastic container during dose preparation. The original DNX-2401 vial and all pipette tips, syringes, needles and needle covers that come into contact with DNX-2401 should be placed in the disinfectant container at the completion of the procedure.

Dosage preparation. Precautions consistent with institutional standards for the handling of viruses (Biosafety Level 2) should be maintained during the preparation and administration of DNX-2401. In addition, standard chemotherapy preparation precautions (gown, gloves, mask and glasses) and sterile technique will be followed while preparing the required dose of DNX-2401. Virus preparation should be performed in a laminar flow biological safety cabinet (LFBSC) using disposable materials. The diluent used for virus dose preparation is USP normal saline.

Once removed from the ≤−60° C. freezer, DNX-2401 should be allowed to thaw completely at room temperature, approximately 5 minutes, but no longer than 15 minutes. Once thawed, if DNX-2401 will not be diluted immediately, it should be placed on ice for a maximum of 2 hours.

Care should be taken to ensure that all the liquid is at the bottom of the vial before removing the flip-off seal, cleansing the sterile vial top and entering the vial. If it is suspected that DNX-2401 is on the sides or top of the vial, the vial can be tapped gently on a solid surface to return the liquid to the bottom of the vial. The vial and associated vessels should be labeled with date and time of thaw and window for use. DNX-2401 must be administered within 3 hours of the time noted for completion of virus preparation.

Caution should be used to avoid excessive shear forces in aspirating and discharging DNX-2401 through needles.

Virus. DNX-2401, Lot #817003 supplied in 2.0 ml vials with a virus concentration in vial of 2.0×10¹¹ PV/mL. The vial volume is 0.4 mL with an extractable volume of 0.2 mL

Transport and dispensing. The prepared DNX-2401 will be transported to the Operating Room in a rigid portable container with ice. The container will have the labels “contains genetically modified organism” and “Biological Hazard”).

The container with the virus will be passed to the OR nurse (sent by the IP) through the OR window of pharmacy department. The nurse will bring to pharmacy the prescription for clinical trial medication signed by the IP or delegated co-investigator. Time of dispensing will be noted. The pharmacist will sign the dispensing. Once preparation is complete, the dose must be administered to the subject within three hours.

Adenovirus infection is a disease that affects pediatric and adult patients in the same degree and with the same virulence, it is not more dangerous in children than in adults and the viral particles during infection is the same. So, for this treatment the inventors do not start with a lower dose because it is a pediatric population. Rather, they are starting in a lower dose because they are injecting the virus in a smaller cavity (pons) instead of the supratentorial brain where there is more room, and previous trials have a majority of cases with supratentorial tumors.

In a previous trial the inventors injected the virus in a pontine tumor with no adverse event and thus think it is justified to start with a lower dose and if no toxicity is detected to escalate to the target dose.

Permitted associated treatments. Administration and dose of corticosteroids, anti-epileptic and antiemetic drugs will be recorded. Corticosteroid therapy will be adapted to the clinical situation of each patient, the minimal necessary dose will be used, and it will be stopped when possible. Antiepileptic drugs will be used only for patients with at least one well-characterized seizure.

Non-permitted associated Treatments. Any other chemotherapy before the virus injection or during the 2 weeks after the injection.

### Example 5—Response Evaluation

Primary Objective. The safety of the DNX-2401 (Delta-24-RGD) will be evaluated using the findings of physical and neurological evaluations, Lansky/Karnofsky Performance Status (KPS), clinical laboratory test results, adverse event (AE)/adverse reaction (AR) occurrences and concomitant medications.

Secondary Objectives. Efficacy will be evaluated by survival and objective tumor response as determined according to RAPNO criteria following clinical evaluation and review of MRI scans. Time to disease progression, progression-free survival at six months (PFS6), median progression-free survival, overall survival at 12 months (OS12) and median overall survival will also be determined.

Quality of life will be measured using PedsQL. If tissue is available and funding is obtained, tumor sequencing will be obtained.

Evaluation of variables—main variable. Neurological status: Follow-up is indicated in section 4.2.3 Complete physical exam, including Lansky/Karnofsky Performance Status and full Neurological exam will be made before and after surgery and in every visit.

Hematologic status: Follow-up is indicated in section 4.2.3 Hematology, Chemistry (including C-reactive protein) and coagulation testing. Glycaemia and coagulation test will be done at inclusion and in every visit.

Any toxicity will be graded according to the National Cancer Institute Common Toxicity Criteria NCI-CTC version 4.03.

Evaluation of variables—secondary variables. Clinical evaluation and MRI with standard sequences will be used to evaluate disease progression, according to RAPNO criteria. In the event of suspicion of progression, advanced MRI sequences could be added at the discretion of the investigator.

Quality of life will be measured using PedsQL. If tumor tissue and funding is available, tumor gene sequencing will be obtained.

Task Distribution. Pediatric Neuro-oncology Area: All the clinical management tasks will be included within this multidisciplinary Area. The Neurosurgeons will be responsible for:
Selection of candidates, and explanation of the trial to the patients to allow them to give informed consent.
Surgical planning, confirmation of entry in the trial, and virus injection.
Clinical Follow-up until hospital discharge after virus injection

Neuro-oncologist or Pediatric oncologists of the Neuro-oncology area will be responsible for:
Follow-up of clinical and laboratory data.
Evaluation of eventual toxicity.
Planning the following best evidence treatment for DIPG.

### Pathology Department:

Keep the tissue in the tumor bank for future studies.
Study of the tumor cells, where the tumor is injected.
Hospital Pharmacy.
Keeping and dilution of the virus dose in the syringe.

Radiology Department: Evaluation of the MRI studies obtained from the patients. Evaluation of radiological response to the therapy. If, for any reason, a patient of the study gets an MRI performed in a center outside the Clinica Universidad de Navarra, it can be accepted for use in the trial after one of the investigators from Radiology Department evaluate it and find the study is of sufficient quality.

Study center: The project will take place in the University Clinic of Navarra.

### Example 6—Adverse Events

All adverse events will be recorded in the CRF and graded according to the NCI-CTC v.4.03. In the event of suspicion of serious toxicity associated to the viral dose, an external safety evaluation Committee will evaluate this toxicity and the trial will be paused until a decision is taken about shifting to a lower dose or other change of the trial.

An Adverse Event (AE) is defined as any event that results in worsening of the health of the subject of the clinical trial, although it has no relationship to the experimental therapy. It can be any symptom, sign, illness or experience, including abnormal results of diagnostic procedures, that develops or worsens in severity during the course of the study.

Serious Adverse Event is defined as any AE that is:
fatal
life-threatening (the concept “life-threatening” means that in the opinion of the investigator, the patient is in real danger of death in the AE situation, it do not mean, that the AE could have caused death if it would have been more intense)
requires or prolongs hospital stay
results in persistent or significant disability or incapacity
a congenital anomaly or birth defect

Any AE that constitute an important medical event will be treated as a SAE regarding notification procedures. Important medical events are those that may jeopardize the subject, and may require intervention to prevent one of the other serious outcomes noted above. All proven incidents of transmission of an infectious agent through the delivery of experimental therapy will also be notified as SAEs.

Adverse reaction is any untoward medical occurrence associated to the administration of a research drug. It different to an AE in that, in the AR, there is a suspicion of causal relationship between the research drug and the effect. The relationship of the causal relationship to the experimental therapy will be established according to the following definitions:

| Relationship | Definition |
|---|---|
| Unrelated | There is no evidence of causal relationship |
| Unlikely | Few data suggest a causal relation, temporal |
| | relationship makes it unlikely, there is another |
| | likely explanation for the event. |
| Possible | There is a reasonable time sequence to administration |
| | of the drug but could also be explained by concurrent |
| | disease or other drugs or chemicals. |
| Probable/Likely | Occurs within a reasonable time sequence to drug |
| | administration, is unlikely to be attributed to |
| | concurrent disease or other drugs or chemicals. |
| Certain | Occurs in a plausible time relationship to drug |
| | administration and cannot be explained by concurrent |
| | disease or other drugs or chemicals. |

The relationship or causality of the AE to therapy (iDNX02401) will be determined by the PI.

Any AE that is classified as Probable or Certain, will be considered as AR. The classification of the causal relationship to the experimental therapy is responsibility of the PI of the center, or the person in whom he delegates.

Suspected Unexpected Serious Adverse Reaction (SUSAR). A SUSAR is a serious adverse reaction (SAR) as noted above, is also suspected and unexpected. Unexpectedness is defined as an adverse event in which the nature, severity, specific or consequences have not been previously observed or noted in the reference information for the drug or that has been noted in the same drug class. An ADR with a fatal outcome is considered unexpected.

Registry of AE. All the AE will be documented, including the ones observed during clinical visits and the ones notified by the patient. Protocol-required follow-up is at least 12 weeks following the virus injection.

The patient should be asked about possible AEs in the time between clinical visits. All AE will be documented in the CRF and in the medical history. All AE will be followed until resolution.

Serious AE that do not require notification to responsible of pharmacovigilance. AE should not be reported as SAE in the following cases:
Hospitalization or prolonged hospitalization for scheduled diagnostic or elective surgical procedures.
Hospitalization or prolonged hospitalization required administering the experimental therapy.
Hospitalization or death because of progression of the target disease of the study.

This event will be documented in the CRF and the medical history of the patient. The rest of the SAE and all cases of pregnancy will be notified following the procedures below.

Procedure of Notification. Notification of SAEs. In case of occurrence of a SAE that must be notified to pharmacovigilance, a SAE Serious Adverse Events Reporting Form must be completed and signed by the investigator and faxed within 24 hours. The investigator will keep a copy of this SAE form on file at the study site.

When additional information about the SAE becomes available, or when the SAE is solved, or when any change is unexpected, the investigator must provide further information in the form of a written narrative that should also be faxed to the pharmacology vigilance responsible. The responsible will review the received form and will ask for additional information if needed. In the event of SUSAR, follow-up information will be provided.

SAE appearing more than 30 days from the end of the experimental therapy will be notified it the investigator consider that there is a causal relationship to the therapy or if it is of medical importance.

Notification of a Pregnancy. If any subject or the partner of a subject becomes pregnant following the administration of DNX-2401, the subject will continue on study at the discretion of the Principal Investigator and be followed regularly until birth or termination of the pregnancy. The pregnancy must be immediately reported to the Sponsor and the owner of the drug license for DNX-2401, DNAtrix. Forms for reporting pregnancies will be provided upon request. The anticipated date of birth or date of termination of the pregnancy should be provided at the time of initial report. The outcome of a pregnancy (for a subject or for the partner of a subject) must be reported to the Medical Monitor, or appropriate designee, as soon as it is known. If the pregnancy ends for any reason before the anticipated date initially reported, the investigator must notify the Sponsor as soon as possible.

If the outcome of the pregnancy meets any criterion for classification as a SAE (including stillbirth, neonatal death, spontaneous abortion, or congenital anomaly-including that in an aborted fetus) the investigator must follow the procedures for reporting SAEs. Any neonatal death occurring ≤30 days after birth will be reported as a SAE.

Notification of Laboratory Abnormalities. All laboratory results must be filed in the subject's medical record and be monitored. The investigator must review laboratory results in a timely manner demonstrated by signature/date and assignment of clinical significance assessment. Non-clinically-significant laboratory abnormalities, i.e., minor deviations from the normal range, are expected and it is likely that no medical intervention will be required. Such results will not be considered to be adverse events.

Clinically Significant (CS) Laboratory Abnormalities. All CTCAE Grade 3 and 4 laboratory result abnormalities and any laboratory abnormality that is considered to be clinically significant by the investigator will be recorded on the AE case report form. An abnormal test result will be considered an AE if:
It is not associated with an already reported AE, diagnosis or pre-existing condition
There is a change in concomitant medication or intervention as needed, in direct response to the Grade 3 or 4 laboratory result
The investigator exercises his/her discretion to make significance determinations for any subject laboratory result

All such laboratory abnormalities will be repeated and assessed by the investigator, or licensed (MD), as soon as possible for “seriousness” and if they meet the regulatory definition of “serious,” they will be reported as SAEs following regulatory and protocol requirements. Repeat laboratory tests may be run in order to monitor the result.

Serious Laboratory Abnormalities. Any laboratory abnormality meeting the regulatory definition of “serious” must be recorded both on the adverse event case report form and the Serious Adverse Event Form. If a subject experiences a serious toxicity or dies, the local and national required regulatory authorities will be notified within 24 hours of awareness of the event, as required. All laboratory values will be statistically analyzed at the end of the study.

Urgent notification of SUSAR to health authorities/Clinical Research Ethics Committee (CEIC). The pharmacovigilance responsible will notify all SUSAR to the AEMPS (Área de ensayos clínicos de la Subdirección Gral. De Medicamentos de Uso Humano) following the procedures approved by law.

Notification Time Limit. Any SUSAR will be reported within 15 calendar days from the moment when the sponsor has knowledge of it. Any SUSAR, fatal or life-threatening, should be notified within 7 days of the moment the sponsor had knowledge of it. The initial information should be completed, if possible, within 8 additional days.

Urgent notification of other relevant safety information. Urgent notification to AEMPS will also be employed for all the information that could modify the risk-to-benefit balance of the experimental therapy or advice changes in the therapy, or in the trial, like, for example:
A change in expected SAR, be in its quality or in its frequency, that is considered clinically relevant.
SUSAR happening after end of follow-up, that the investigator notifies to the sponsor who then notifies applicable regulatory authorities.
New events related to the trial or to the IND development that could affect to the safety like:
   SAEs that can be related to the study procedures and advice to modify it.
   A significant risk to the subjects, like the lack of efficacy of a drug used in a life-threatening disease.
   New significant findings about the safety of the drug from animal studies.
   Any premature termination or pause for safety reason in a trial with the same drug in another country.

Regulatory authorities will be notified of the new information will be notified as soon as possible and no later than 15 days from when the investigator has knowledge of it. Additional information will be provided also as soon as possible.

Annual safety report. All SUSAR and SAEs reported will be notified to the AEMPS (Área de Ensayos clínicos de la Subdirección General de Medicamentos de Uso Humano in an annual safety report) and to the CEIC.

Notifications to investigators. This study is unicentric without investigators at other centers who would require notification. Any safety information possibly involving the subjects of the trial will be notified as soon as possible to the investigators at the single participating site. Information about SUSARs will be sent annually, compiled in a listing with a short analysis of the data. Information about SUSARs will be provided to applicable regulatory authorities annually, compiled in a listing with a short analysis of the data. The Principal Investigator will provide the national regulatory authorities with an annual report.

Any safety aspect that can impact on the clinical trial or in the development of the IND, including stopping the development program of the drug, or protocol changes for safety, will also be notified to the investigator.

Trial drug discontinuation due to adverse events. Administration of DNX-2401 occurs only once. Once administered, it cannot be discontinued. The Principal Investigator can stop the future enrollment of study participants if there are frequent and consistent safety concerns.

### Example 7—Statistical Analyses

The sample size was estimated according to a binomial test. For a sample size of 12 patients, a power of 88.5% is obtained to detect the difference of 0.45 between the alternative and null values (Current mortality rate within 12 months of presentation: 70%), with an observed significance level of 0.041, assuming a 5% dropout rate.

Brain tumors have an incidence of 3.3 per 100,000 children and DIPG are around 5-7% of all brain tumors. Every year we would expect a DIPG incidence of 0.2 per 100.000 children. In 2015, there were 6 million children between 0-15 years in Spain (world-wide-web at ine.es), which seems warrant the ability to recruit the number of children with DIPG required for this study.

The study will include only patients diagnosed of DIPG and that have not received any other previous treatment.

All available data about safety and efficacy will be tabulated. No imputation of unavailable data will be made. Any eventual confuse or mistaken data will be managed according to standard procedures.

For toxicity and safety, descriptive statistic will be used. For secondary objectives, PFS6 and OS12 will be compared with the current reference value using a binomial test. Data will be analyzed using Stata 14 software (StataCorp. 2015. Stata Statistical Software: Release 14. College Station, TX: StataCorp LP). The specified significance level is 0.05.

Safety evaluation will depend upon incidence, severity and kind of adverse events. Safety data from all the patients will be tabulated.

All the AE observed during the study will be included in a relation organized by patient. Those AEs that are possible, probable or certain to be associated to the experimental therapy will be included in a specific table.

AEs will also be organized and presented by severity. All deaths, SAE and SAEs resulting in suspension of therapy will also be presented separately.

All deviations from the planned analysis would be reflected in the final report of the study.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 8—Clinical Study Results

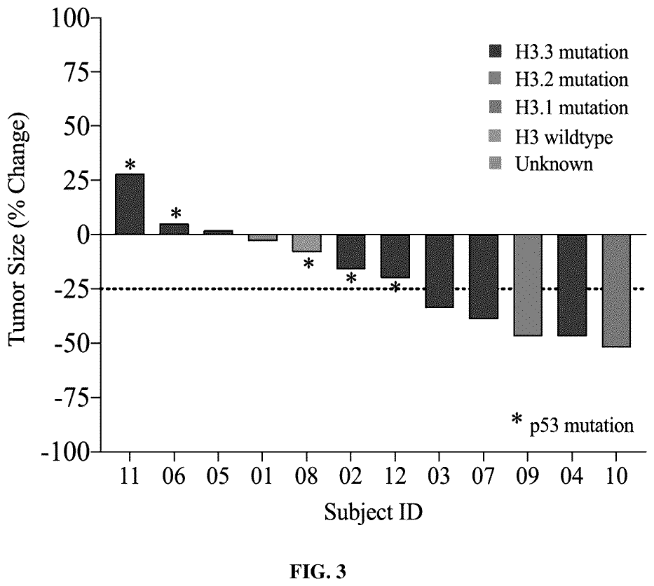

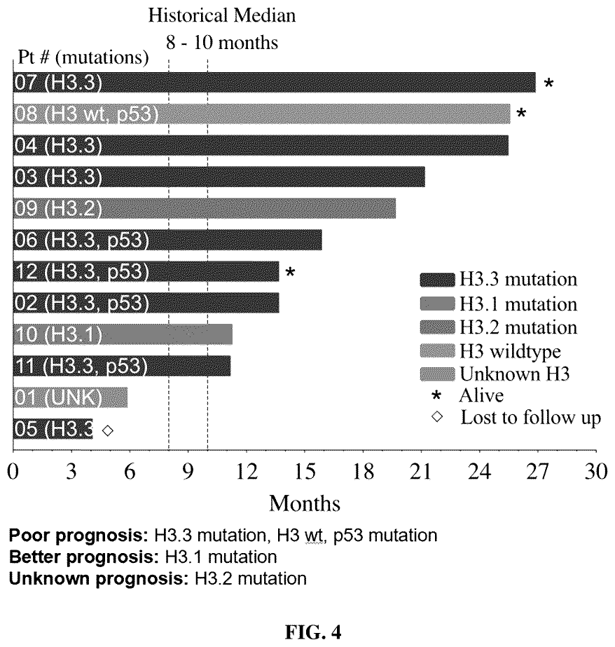

Summary A phase 1 study (full protocol, Example 1) is being conducted to evaluate the safety and efficacy of a single intratumoral injection of DNX-2401 followed by conventional radiation therapy in pediatric subjects with newly diagnosed DIPG. Primary and secondary endpoints include safety, efficacy, OS-12, quality of life, and tumor molecular profiling. From 1 Dec. 2017 to 24 Jan. 2020, 12 subjects aged 3-18 (median 9) without prior treatment for DIPG were enrolled with Lansky/Karnofsky performance scores of 70-80 (n=8; 66.7%) or 90-100 (n=4; 33.3%). A tumor biopsy sample was collected immediately prior to a single intratumoral dose of 1e10 vp (n=4, subjects 01-04) or 5e10 vp DNX-2401 (n=8, subjects 05-12), which was then followed by standard radiation therapy approximately 2-4 weeks later (11 of 12; 91.7%). The most commonly reported adverse events regardless of drug relationship have included primarily grade 1-2 headache, asthenia, vomiting, anemia, leukocytosis, and fever. Maximum percent tumor change was calculated by assessing percent change within 3 months of initiating treatment, compared to baseline MRIs per RAPNO criteria. 25% or more reduction sustained for 8 weeks was utilized to determine clinical response. Tumor reductions have been observed, including >25% decrease for 5 subjects (41.7%) per RAPNO criteria (FIG. 3; Cooney 2020). Mutational status at the time of study entry for histone H3 and p53 were determined from biopsy material. Survival was calculated from the start of treatment to death or last follow-up. Ten (1)) of 12 subjects (83%) have survived at least 10 months, including 3 subjects reaching 24 months (25.0%; FIG. 4), to date. Three subjects (25%) are being followed for survival (26.9, 24.7, 12.9 months) and preliminary survival estimates per Kaplan-Meier analysis include a median of 19.7 months, OS-12 of 72.7%, and OS-24 of 32%. The unexpectedly long median survival and survival at 12 and 24 months significantly exceed the historical median survival range for newly diagnosed DIPG based on published studies of 8-10 months, OS-12 of 26-40%, and OS-24 of 4-7% (Mandell 1999, Zaghloul 2014, Cohen 2011).

Subjects with poor prognostic factors. Particularly surprising results of treatment of DIPG with DNX-2401 were the tumor reductions and promising survival observed in subjects with age and genetic factors associated with poor survival and resistance to radiation (H3.3 mutation, H3 wildtype, p53 mutation; FIG. 4). All subjects were 3 years of age or older and 8/12 subjects were within the worst prognosis age group, between 3 and 9 years of age. 10/12 subjects with documented H3.3 K27M, H3 wildtype, and/or p53 mutation survived longer than the historical median despite having one or more of these poor prognostic genetic mutations. Median and landmark survival of subjects with H3.3 K27M mutation was 21.2 months, with OS-12 of 86%, and OS-24 of 36%. Similarly, subjects with p53 mutations typically associated with poor response to radiation had median and landmark survival of 16 months, with OS-12 of 80%, and OS-24 of 30% suggesting that treatment with DNX-2401 was able to synergize with or overcome resistance to radiation in these subjects.

Quality of life and reduction in steroid use. It was not previously known whether delivery of an adenovirus to the pons, particularly in small children, would be tolerated. Both the volume of fluid and inflammatory properties of the virus have the potential to cause significant edema. Surprisingly, all subjects tolerated the delivery well with no requirement for high dose steroid to alleviate edema. In fact, all subjects were free of all steroid by 4 weeks post treatment, and all showed improvements in quality of life parameters after 4 weeks. This finding in pediatric subjects is unexpected as 22% of adult subjects with glioblastoma treated with DNX-2401 experienced cerebral edema.

Preclinical Models of DIPG. In preclinical models of DIPG, Delta-24-RGD has direct oncolytic activity and elicits antitumor immune responses including infiltration of CD4+ and CD8+ T-cells into tumors post-treatment (Martinez-Velez 2019a). The combination of Delta-24-RGD and RT results in synergistic anti-tumor effects in vitro and increased survival in models of DIPG including those with unfavorable histone mutations (Martinez-Velex 2019b). Like DIPG, AT/RTs and CNS-PNETs are pediatric brain tumors with few therapeutic options at recurrence and a poor survival rate. However their molecular profile is different, with AT/RTs characterized by mutations in chromatin remodeling proteins, and CNS-PNET presenting as a heterogeneous group with yet to be determined molecular markers. The efficacy of administration of Delta-24-RGD was evaluated in different orthotopic mouse models of AT/RT and CNS-PNET, including infratentorial and supratentorial models (Garcia-Moure 2020). A single intratumoral Delta-24-RGD injection (10⁷ or 10⁸ PFU) significantly increased survival and led to long-term survival in both AT/RT and PNET models. Delta-24-RGD hindered dissemination of AT/RTs and increased survival, leading to 70% of long-term survivors. Of relevance, viral administration to established tumor masses (30 days after engraftment) showed therapeutic benefit. In humanized immunocompetent models (hCD34+-NSG-SGM3), Delta-24-RGD significantly extended the survival of mice bearing AT/RTs or PNETs (ranging from 11 to 27 days) and did not display any toxicity associated with inflammation. Immunophenotyping of Delta-24-RGD-treated tumors revealed increased CD8+ T cell infiltration.

### V. Various Embodiments

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y,” and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The following are some exemplary numbered embodiments of the present disclosure.
1. A method of treating pediatric brain tumors comprising administering to said subject a replication competent oncolytic adenovirus. Within certain embodiments, the brain tumor is a glioma.
2. The method according to embodiment 1 wherein said pediatric brain tumor is Diffuse Intrinsic Pontine Glioma. Within other embodiments the pediatric tumor is a pediatric high-grade glioma, Atypical Teratoid/Rhabdoid Tumor (“AT/RT”), or a Primitive Neuroectodermal Tumor (“CNS-PNET”).
3. The method according to embodiments 1 or 2 wherein said subject has an H3.3 K27M mutation. Within other embodiments, the subject has a H3.3K27M, H3.1K27M, H3.2K27M, wild type H3, H3.3G34RV, and/or p53 mutation (see generally, Mackay et al., 2017 Cancer Cell 32:520-537, Oct. 9, 2017, which is incorporated by reference in its entirety).
4. The method of embodiments 1, 2, or 3 further comprising the step of administering radiation therapy to said patient.
5. The method of embodiments 1, 2, 3, or. 4, wherein no resection surgery is performed on the patient.
6. The method of embodiments 1, 2, 3, 4, or 5, wherein a neuroventricular catheter is utilized to administer the replication competent oncolytic adenovirus.
7. The method of embodiments 1, 2, 3, 4, 5, or 6, wherein the administration of the replication competent oncolytic adenovirus comprises more than one administration, such as two administration, such as two administrations spaced two weeks apart, such as about four weeks prior to surgery and about two weeks prior to surgery.
8. The method of any preceding embodiment, wherein the adenovirus comprises a deletion in part or all of the E1 gene region.
9. The method of embodiment 8, wherein the adenovirus comprises an insertion of an integrin binding motif in the H1 loop of the fiber.
10. The method of embodiment 9, wherein the insertion comprises the peptide CDCRGDCFC.
11. The method of any preceding embodiment, wherein the adenovirus is a human adenovirus type 5 or a hybrid comprising a human adenovirus type 5 component.
12. The method of embodiment 11, wherein the adenovirus is Delta-24 or Delta-24-RGD.
13. The method of any one of embodiments 1-12, wherein the replication competent oncolytic adenovirus is DNX-2401.
14. The method of any one of embodiments 1-13, wherein the adenovirus genome comprises one or more heterologous nucleic acid sequences encoding a tumor antigen, whereby the adenovirus expresses the tumor antigen(s) on its surface.
15. The method of embodiment 14 wherein the tumor antigen is selected from the group consisting of: MAGE-1, MAGE-2, MAGE-3, CEA, Tyrosinase, midkin, BAGE, CASP-8, β-catenin, CA-125, CDK-1, ESO-1, gp75, gplOO, MART-1, MUC-1, MUM-1, p53, PAP, PSA, PSMA, ras, trp-1, HER-2, TRP-1, TRP-2, IL13Rα, IL13Rα2, AIM-2, AIM-3, NY-ESO-1, C9orfl l2, SART1, SART2, SART3, BRAP, RTN4, GLEA2, TNKS2, KIAA0376, ING4, HSPH1, C13orf24, RBPSUH, C6orfl53, NKTR, NSEP1, U2AF1L, CYNL2, TPR, SOX2, GOLGA, BMI1, COX-2, EGFRvIII, EZH2, LICAM, Livin, Livin, MRP-3, Nestin, OLIG2, ART1, ART4, B-cyclin, Glil, Cav-1, cathepsin B, CD74, E-cadherin, EphA2/Eck, Fra-1/Fosl 1, GAGE-1, Ganglioside/GD2, GnT-V, β1,6-N, Ki67, Ku70/80, PROX1, PSCA, SOX10, SOX11, Survivin, UPAR and WT-1 or an immunogenic peptide thereof.
16. The method of embodiment 15, wherein the heterologous nucleic acid is inserted in hyper-variable region 5 of the hexon gene of the adenovirus or is inserted into the HI loop region of the adenovirus fiber gene.
17. The method of embodiment 16, wherein the adenovirus comprises a heterologous nucleic acid encoding EGFRvIII or an immunogenic peptide thereof inserted into the HI loop region of the fiber gene of the adenovirus and/or a heterologous nucleic acid encoding NY-ESO-1 or an immunogenic peptide thereof inserted in the hyper-variable region 5 of the hexon gene of the adenovirus.
18. The method of any one of embodiments 1-13, wherein the adenovirus genome comprises one or more heterologous nucleic acid sequences encoding an immune modulator, whereby the immune modulator is secreted from an adenovirus-infected cell or expressed on the surface of the adenovirus-infected cell.
19. The method of embodiment 18 wherein the immune modulator is a co-stimulatory molecule, e.g., OX40 ligand, CD40 Ligand
20. The method of any one of embodiments 1-12, wherein the replication competent oncolytic adenovirus is DNX-2440.
21. The method of embodiment 18 wherein the immune modulator is a cytokine, e.g., IL-2, IL-12
22. The method of any preceding embodiment, further comprising administering a second anti-cancer therapy, such as chemotherapy, radiotherapy, immunotherapy, hormonal therapy or toxin therapy.
23. The method of embodiments 1-18, further comprising administering to said subject one or more Th1 stimulating agents.
24. The method of embodiment 19 wherein said Th1 stimulating agent is selected from the group consisting of: IL-12p70, IL-2, IFN-γ, lenalidomide, temozolomide (4-methyl-5-oxo-2,3,4,6,8-pentazabicyclo [4.3.0] nona-2,7,9-triene-9-carboxamide), cyclophosphamide ((RS)-N,N-bis(2-chloroethyl)-1,3,2-oxazaphosphinan-2-amine 2-oxide), lomustine (CCNU; N-(2-chloroethyl)-N′-cyclohexyl-N-nitrosourea), bis-chloroethylnitrosourea (BCNU), melphalan hydrochloride (4-[bis(chloroethyl)amino]phenylalanine), busulfan (butane-1,4-diyl dimethanesulfonate), mechlorethamine (nitrogen mustard), chlorambucil, ifosfamide, streptozocin, dacarbazine (DTIC), thiotepa, altretamine (hexamethylmelamine), cisplatin, carboplatin, oxalaplatin, Ipilimumab, Tremelimumab, MDX-1106, MK-3475 (pembrolizumab), AMP-224, Pidilizumab, and MDX-1105.
25. The method of any preceding embodiment, wherein the replication competent oncolytic adenovirus is administered systemically, local or regional to said tumor, or intratumorally
26. The method of any of the preceding embodiments wherein the replication competent oncolytic adenovirus is administered, intravascularly, or in a neuronal or mesenchymal stem cell carrier.
27. The method of any one of the preceding embodiments wherein the replication competent oncolytic adenovirus is administered intratumorally or into the tumor vasculature.
28. The method of embodiment 24, wherein administration comprises MR-guided administration, such as with a neuroventricular cannula.
29. The method of any one of the preceding embodiments wherein the replication competent oncolytic adenovirus is administered once or multiple times at a dose of 10⁸-10¹³ plaque forming units (pfu).
30. The method of embodiment 26, whereby tumor growth is reduced in the treated tumor
31. The method of embodiment 26, whereby tumor size is reduced in the treated tumor.
32. The method of any one of the preceding embodiments wherein the subject is a human.

1. A method of treating pediatric brain tumors comprising administering to said subject a replication competent oncolytic adenovirus.

2. The method of claim 1, wherein said pediatric brain tumor is Diffuse Intrinsic Pontine Glioma.

3. (canceled)

4. The method of claim 2, wherein said subject has one or more of a H3.3 K27M mutation, a H3.1 K27M, a H3.2 K27M mutation and/or a wild-type H3 mutation.

5. The method of claim 1, further comprising administering radiation therapy to said subject.

6. The method of claim 1, wherein no resection surgery is performed on the subject.

7. The method of claim 1, wherein a neuroventricular catheter is utilized to administer the replication competent oncolytic adenovirus.

8. The method of claim 7, wherein the replication competent oncolytic adenovirus is administered in a single administration or multiple administrations at a dose of 10⁸-10¹³ plaque forming units (pfu).

9. The method of claim 1, wherein the adenovirus comprises a deletion in part or all of a E1 gene region.

10. The method of claim 9, wherein the adenovirus comprises an insertion of an integrin binding motif in a H1 loop of a fiber.

11. The method of claim 10, wherein the insertion comprises a peptide CDCRGDCFC.

12. The method of claim 1, wherein the adenovirus is a human adenovirus type 5 or a hybrid comprising a human adenovirus type 5 component.

13. The method of claim 1, wherein the replication competent oncolytic adenovirus is selected from Delta-24, Delta-24-RGD, DNX-2401 or DNX-2440.

14. (canceled)

15. The method of claim 1, wherein a genome of the adenovirus comprises one or more heterologous nucleic acid sequences encoding a tumor antigen, whereby the adenovirus expresses the tumor antigen(s) on a surface of an adenovirus-infected cell.

16. The method of claim 15, wherein the tumor antigen is selected from the group consisting of: MAGE-1, MAGE-2, MAGE-3, CEA, Tyrosinase, midkin, BAGE, CASP-8, β-catenin, CA-125, CDK-1, ESO-1, gp75, gplOO, MART-1, MUC-1, MUM-1, p53, PAP, PSA, PSMA, ras, trp-1, HER-2, TRP-1, TRP-2, IL13Rα, IL13Rα2, AIM-2, AIM-3, NY-ESO-1, C9orfl 12, SART1, SART2, SART3, BRAP, RTN4, GLEA2, TNKS2, KIAA0376, ING4, HSPH1, C13orf24, RBPSUH, C6orf153, NKTR, NSEP1, U2AF1L, CYNL2, TPR, SOX2, GOLGA, BMI1, COX-2, EGFRvIII, EZH2, LICAM, Livin, Livin, MRP-3, Nestin, OLIG2, ART1, ART4, B-cyclin, Glil, Cav-1, cathepsin B, CD74, E-cadherin, EphA2/Eck, Fra-1/Fosl 1, GAGE-1, Ganglioside/GD2, GnT-V, β1,6-N, Ki67, Ku70/80, PROX1, PSCA, SOX10, SOX11, Survivin, UPAR and WT-1 or an immunogenic peptide thereof.

17. The method of claim 16, wherein a heterologous nucleic acid is inserted in hyper-variable region 5 of a hexon gene of the adenovirus or is inserted into a HI loop region of a fiber gene of the adenovirus.

18. The method of claim 17, wherein the adenovirus comprises a heterologous nucleic acid encoding EGFRvIII or an immunogenic peptide thereof inserted into the HI loop region of the fiber gene of the adenovirus and/or a heterologous nucleic acid encoding NY-ESO-1 or an immunogenic peptide thereof inserted in the hyper-variable region 5 of the hexon gene of the adenovirus.

19. The method of claim 1, further comprising administering a second anti-cancer therapy selected from chemotherapy, radiotherapy, immunotherapy, hormonal therapy or toxin therapy.

20. The method of claim 1, further comprising administering to said subject one or more Th1 stimulating agents, wherein said one or more Th1 stimulating agents are selected from the group consisting of: IL-12p70, IL-2, IFN-γ, lenalidomide, temozolomide (4-methyl-5-oxo-2,3,4,6,8-pentazabicyclo [4.3.0] nona-2,7,9-triene-9-carboxamide), cyclophosphamide ((RS)-N,N-bis(2-chloroethyl)-1,3,2-oxazaphosphinan-2-amine 2-oxide), lomustine (CCNU; N-(2-chloroethyl)-N′-cyclohexyl-N-nitrosourea), bis-chloroethylnitrosourea (BCNU), melphalan hydrochloride (4-[bis(chloroethyl)amino]phenylalanine), busulfan (butane-1,4-diyl dimethanesulfonate), mechlorethamine (nitrogen mustard), chlorambucil, ifosfamide, streptozocin, dacarbazine (DTIC), thiotepa, altretamine (hexamethylmelamine), cisplatin, carboplatin, oxalaplatin, Ipilimumab, Tremelimumab, MDX-1106, MK-3475 (pembrolizumab), AMP-224, Pidilizumab, and MDX-1105.

21. (canceled)

22. The method of claim 1, wherein the replication competent oncolytic adenovirus is administered systemically, local or regional to a tumor, intratumorally, intravascularly, in a neuronal or mesenchymal stem cell carrier, into a tumor vasculature.

23.-26. (canceled)

27. The method of claim 1, wherein tumor growth and/or tumor size is reduced in the treated tumor.

28. (canceled)

29. (canceled)
